(19) United States
(12) Patent Application Publication
(10) Pub. No.: US 2007/0225382 A1
(43) Pub. Date: Sep. 27, 2007

(54) Method for producing synthesis gas or a hydrocarbon product

(76) Inventors: Robert Erwin Van Den Berg, Amsterdam (NL); Johannes Margaretha Anna Joan Van Montfort, Amsterdam (NL); Jacobus Hendrikus Scheerman, Amsterdam (NL); Johannes Gerardus Maria Schilder, Amsterdam (NL)

Correspondence Address: SHELL OIL COMPANY, P O BOX 2463, HOUSTON, TX, 772522463, US

(21) Appl. No.: 11/548,987

(22) Filed: Oct. 12, 2006

(30) Foreign Application Priority Data
Oct. 14, 2005 (EP) 05109559.4

(51) Int. Cl. C10J 3/00; C07C 27/06; C07C 27/00

(52) U.S. Cl. 518/702; 48/210

(57) ABSTRACT

Synthesis gas is produced from a carbonaceous fuel, using a process in a gasification reactor. The carbonaceous fuel and an oxygen containing stream are supplied to a burner of a gasification reactor, wherein a CO₂-containing transport gas is used to transport the solid carbonaceous fuel to the burner. The weight ratio of CO₂ to the carbonaceous fuel is less than 0.5 on a dry basis. The carbonaceous fuel is partially oxidized in the gasification reactor, whereby a gaseous stream of synthesis gas is obtained. This synthesis gas can be further processed in a downstream process path to convert it into a hydrocarbon product. The downstream process path may contain a methanol-synthesis reactor to produce the hydrocarbon product in the form of methanol. The downstream process path may contain a Fischer-Tropsch synthesis reactor to produce one or more from a range of hydrocarbon products.

### FIELD OF THE INVENTION

The present disclosure is directed to a process for producing synthesis gas or a hydrocarbon product from a carbonaceous fuel.

### BACKGROUND OF THE INVENTION

Synthesis gas typically comprises carbon-monoxide (CO) and hydrogen (H₂).

Various methods are known for the production of synthesis gas or a hydrocarbon product from a carbonaceous fuel.

Gasification of solid carbonaceous fuels such as coal is well known, and often involves milling or otherwise grinding the fuel to a preferred size or size range, followed by reacting the fuel with oxygen in a gasifier. This creates the mixture of hydrogen and carbon monoxide referred to as syngas or synthesis gas.

A specific example of a process for the production of synthesis gas and methanol from coal is described in a paper by M. J. van der Burgt and J. E. Naber, entitled <<Development of the Shell Coal Gasification Process>> (published in the proceedings of the third BOC Priestley Conference, held in September 1983 in London), incorporated herein by reference. In the described system and process, ground and dried coal is pressurized in a lock-hopper and pneumatically fed to a gasification reactor where it is converted into a gaseous fuel base material by reacting with a blast containing oxygen and steam or air. The gaseous fuel base material is fed to a downstream system including a CO shift convertor, CO₂ removal, and a methanol synthesis reactor.

The Fischer-Tropsch process is another process that can be used for the conversion of hydrocarbonaceous feedstocks into liquid and/or solid hydrocarbon products. The feedstock (e.g. natural gas, associated gas, coal-bed methane, biomass, heavy oil residues, coal) is converted in a first step into a mixture of hydrogen and carbon monoxide (this mixture is often referred to as synthesis gas or syngas). The synthesis gas is then fed into a reactor where it is converted over a suitable catalyst at elevated temperature and pressure into paraffinic compounds ranging from methane to high molecular weight molecules comprising up to 200 carbon atoms, or, under particular circumstances, even more. Examples of the Fischer-Tropsch process are described in e.g. U.S. Pat. No. 6,759,440, WO-A-01/76736, US Application Publication No. 2003/0181535, EP-A-510771 and EP-A-450861, each of which is incorporated herein by reference.

Numerous types of reactor systems have been developed for carrying out the Fischer-Tropsch reaction. For example, Fischer-Tropsch reactor systems include fixed bed reactors, especially multi-tubular fixed bed reactors, fluidised bed reactors, such as entrained fluidised bed reactors and fixed fluidised bed reactors, and slurry bed reactors such as three-phase slurry bubble columns and ebulated bed reactors.

In known gasification processes, N₂ is used as a transport gas for transporting the carbonaceous fuel, especially if ammonia is one of the intended products.

Although relatively inert, such use of N₂ as a transport gas in the production of synthesis gas may lead to undesirably reducing the catalytic efficiency of downstream processes. This is even more pertinent if the process is especially intended for producing a hydrocarbon product not containing N-atoms.

In particular, nitrogen has been found to adversely affect a hydrocarbon-forming reaction using a catalytic process, including methanol-forming reactions or Fischer-Tropsch type reactions.

Moreover, the presence of nitrogen will require more reactor volume for performing the hydrocarbon-forming reaction at the same production capacity, including for synthesis of methanol and Fischer-Tropsch synthesis. This is especially the case when a synthesis gas recycle over the reactor is applied.

EP-A-444684 describes a process to prepare methanol from solid waste material. In this process a solid waste is combusted at ambient pressure with oxygen and a stream of carbon dioxide. The combustion takes place in a furnace to which solid waste material is supplied from the top and the oxygen and carbon dioxide streams from the bottom. Carbon dioxide is added because it serves as a methanol building block and to suppress the temperature in the furnace. The synthesis gas as prepared in the furnace is used to make methanol. Part of the carbon dioxide present in the synthesis gas is recycled to the furnace.

The furnace in the process of EP-A-444684 is operated at ambient pressure. When desiring a high capacity, especially when starting from a solid coal fuel, large furnaces will be required.

A process, which is operated at higher pressure, is described in U.S. Pat. No. 3,976,442. In this publication a solid carbonaceous fuel is transported in a CO₂ rich gas to a burner of a pressurized gasification reactor operating at about 50 bar. According to the examples of this publication a flow of coal and carbon dioxide at a weight ratio of CO₂ to coal of about 1.0 is supplied to the annular passage of the annular burner at a velocity of 150 ft/sec. Oxygen is passed through the centre passage of the burner at a temperature of 300° F. and a velocity of 250 ft/sec. U.S. Pat. No. 3,976,442 thus provides a process wherein the partial oxidation is performed in a pressurized reactor and wherein the use of nitrogen as transport gas is avoided.

Nevertheless the use of carbon dioxide as transport gas was never practiced or seriously considered in the intermediate 30 years. This was probably due to the low efficiency of the process as disclosed by this publication. The low efficiency of synthesis gas production will ultimately also effect the efficiency of the total process starting from the carbonaceous fuel to the products as obtained in the downstream conversions including hydrocarbon product syntheses including methanol-synthesis and Fischer-Tropsch synthesis.

### SUMMARY OF THE INVENTION

The present invention provides a process for producing synthesis gas or a hydrocarbon product from a carbonaceous fuel, the process comprising the steps of:

(a) supplying a carbonaceous fuel and an oxygen containing stream to a burner of a gasification reactor, wherein a CO₂ containing transport gas is used to transport the carbonaceous fuel to the burner, whereby the weight ratio of CO₂ to the carbonaceous fuel is less than 0.5 on a dry basis;

(b) partially oxidising the carbonaceous fuel in the gasification reactor, thereby obtaining a gaseous stream at least comprising CO, CO₂, and H₂; and

(c) removing the gaseous stream obtained in step (b) from the gasification reactor.

The gaseous stream may be further processed. For instance, the gaseous stream may be led into a hydrocarbon reactor system to be transformed into a hydrocarbon product. The further processing of the gaseous stream may also be aimed at producing H₂.

### BRIEF DESCRIPTION OF THE DRAWINGS

Hereafter the present invention will be further illustrated by way of example, with reference to the following non-limiting drawings in which:

FIG. 1 schematically shows a process block scheme or flow diagram of a hydrocarbon-product synthesis system including a fuel supply system and a gasification system and a downstream part of the process;

FIG. 2 schematically shows a process block scheme or flow diagram of a detailed embodiment for carrying out a downstream part of the process; and

FIG. 3 schematically shows a process block scheme or flow diagram of a detailed further embodiment for carrying out the downstream part of the process.

In the Figures like reference signs relate to like components.

### DETAILED DESCRIPTION

Embodiments of the present invention employ a CO₂-containing transport gas to transport the carbonaceous fuel to the burner, whereby the weight ratio of CO₂ to the carbonaceous fuel is less than 0.5 on a dry basis.

It has been found that by using such a relatively low weight ratio of CO₂ to the carbonaceous fuel, less oxygen is consumed during the gasification process and a higher selectivity to carbon monoxide and hydrogen is achieved as compared to the process of U.S. Pat. No. 3,976,442. This enhances the total efficiency of the process according to the present invention significantly.

Due to the present dense phase feeding of the carbonaceous fuel to the gasification reactor, the CO₂ content in the gaseous stream is lower than in U.S. Pat. No. 3,976,442. Thus, less CO₂ has to be removed from the process afterwards, than if a dilute phase is used for transporting the carbonaceous fuel to the gasification reactor.

Moreover, for a given amount of carbonaceous fuel to be partially oxidised in the gasification reactor, a smaller reactor volume can be used, resulting in lower equipment expenses.

The gasification process produces a gaseous stream, which may be fed into a downstream system for further processing. Further processing may include filtering out dry solids, modifying the composition of the gaseous stream, and/or separating an H₂ stream as the final product. In other embodiments, the further processing includes feeding into a hydrocarbon synthesising reactor system to form a hydrocarbon product out of the gaseous stream.

For the present disclosure, the term hydrocarbon product is intended to include any hydrocarbon product, inducing alkanes, oxygenated alkanes, and hydroxygenated alkanes such as alcohols, in particular methanol or dimethyl ether (DME).

Thus, embodiments of the present invention may comprise steps of

(a) supplying the carbonaceous fuel and the oxygen containing stream to the burner of the gasification reactor, wherein a CO₂ containing transport gas is used to transport the carbonaceous fuel to the burner;

(b) partially oxidising the carbonaceous fuel in the gasification reactor, thereby obtaining a gaseous stream at least comprising CO, CO₂, and H₂;

(c) removing the gaseous stream obtained in step (b) from the gasification reactor;

(d) optionally shift-converting at least part of the gaseous stream as obtained in step (c) to obtain a CO-depleted stream; and

(e) optionally subjecting the gaseous stream of step (c) and/or the optional CO-depleted stream of step (d) to a hydrocarbon-product synthesising reaction to obtain a hydrocarbon product.

Optionally, carbon dioxide is separated from the gaseous stream prior to performing optional step (e).

The hydrocarbon-product synthesising reaction may be of any suitable type, including

a (catalytic) alcohol-synthesizing reaction such as a methanol-synthesizing reaction or a catalytic methanol-synthesizing reaction;

a Fischer-Tropsch reaction.

The carbonaceous fuel may be any carbonaceous feedstock, including hydro-carbonaceous fuels, preferably in solid form. Examples of solid carbonaceous fuels are coal, brown coal, coke from coal, petroleum coke, soot, biomass, including peat, and particulate solids derived from oil shale, tar sands and pitch. Coal is particularly preferred, and may be of any type, including lignite, sub-bituminous, bituminous and anthracite. All such types of feedstock have different levels of ‘quality’, including the specific proportions of hydrogen and carbon, as well as substances regarded as ‘impurities’ of which sulphur and sulphur-containing compounds, nitrogen-containing compounds, ash, heavy metals form typical examples.

The CO₂-containing transport gas supplied in step (a) may be any suitable CO₂ containing stream. Typically the stream may contain at least 80% CO₂, and preferably at least 95% CO₂. The CO₂-containing transport gas is suitably obtained from a processing step that is performed on the gaseous stream as removed in step (c), later on in the process.

The CO₂ containing stream supplied in step (a) may be supplied at a velocity of less than 20 m/s, preferably from 5 to 15 m/s, more preferably from 7 to 12 m/s. Further it is preferred that the CO₂ and the carbonaceous fuel are supplied as a single stream, preferably at a density of from 300 to 600 kg/m³, preferably from 350 to 500 kg/m³, more preferably from 375 to 475 kg/m³.

In certain embodiments of the present process, the weight ratio in step (a), on a dry basis, is less than 0.50 or preferably below 0.49, more preferably below 0.40, and still more preferably below 0.30 or below 0.20. It may be in a range from 0.12-0.49, and most preferably in the range from 0.12-0.20 on a dry basis.

The gaseous stream obtained in step (c) may comprise, on a dry basis, from 1 to 10 mol % CO₂, preferably from 4.5 to 7.5 mol % CO₂ when performing the process using high-density carbonaceous fuel feeding such as outlined above.

The streams supplied in step (a) may have been pre-treated, if desired, before being supplied to the gasification reactor.

Moreover, the gaseous stream as obtained in step (c) may be further processed. As an example, the gaseous stream as obtained in step (c) may be subjected to dry solids removal, wet scrubbing, etc. Preferably the gaseous stream as obtained in step (c) is subjected to a hydrocarbon synthesis reactor thereby obtaining a hydrocarbon product, in particular methanol.

The shift conversion in step (d) may involve converting CO into CO₂ to obtain the CO-depleted stream. This may be performed in a shift conversion reactor or a sour shift reactor. Typically water, usually in the form of steam, is mixed with the gaseous stream obtained from step (c) to form CO₂ and H₂. A catalyst may be used, and may be selected from any catalyst for such a reaction, including iron, chromium, copper and zinc and combinations thereof. Copper on zinc-oxide is a known suitable shift catalyst.

Step (d) of the process may further comprise subjecting the CO-depleted stream to a CO₂ recovery system, thereby obtaining a CO₂ rich stream and a CO₂-poor CO-depleted stream. The latter may be used in the optional subsequent step (e).

It is preferred to recover at least 80 vol %, preferably at least 90 vol %, more preferably at least 95 vol % and at most 99.5 vol %, of the carbon dioxide present in the CO-depleted stream. This avoids the build-up of inerts in a subsequent hydrocarbon-product synthesizing process of optional step (e).

The CO₂ recovery system may be provided in the form of a combined CO₂/H₂S removal system and/or assist in the removal or reduction of other contaminants such as for instance HCN, NH₃ and COS.

The CO₂ rich stream may at least be partially used as the CO₂ containing transport gas stream for step (a). Excess CO₂ is preferably stored in subsurface reservoirs or more preferably used for enhanced oil or gas recovery or enhanced coal bed methane recovery.

The removal system may use a chemical and/or a physical solvent process and it may involve one or more removal units. A removal unit may be located downstream of optional step (e), such as to remove CO₂ from the off-gas that is separated from the hydrocarbon product as obtained in step (e).

There are chiefly two categories of absorbent solvents on an industrial scale, depending on the mechanism to absorb acidic components: chemical solvents and physical solvents. Each solvent has its own advantages and disadvantages as to features as loading capacity, kinetics, regenerability, selectivity, stability, corrosivity, heat/cooling requirements, etc.

Chemical solvents which have proved to be industrially useful are primary, secondary and/or tertiary amines derived alkanolamines. The most frequently used amine are derived from ethanolamine, especially monoethanol amine (MEA), diethanolamine (DEA), triethanolamine (TEA), diisopropanolamine (DIPA) and methyldiethanolamine (MDEA).

Physical solvents which have proved to be industrially suitable are cyclo-tetramethylenesulfone and its derivatives, aliphatic acid amides, N-methylpyrrolidone, N-alkylated pyrrolidones and the corresponding piperidones, methanol, ethanol and mixtures of dialkylethers of polyethylene glycols.

One suitable known commercial process uses an aqueous mixture of a chemical solvent, especially DIPA and/or MDEA, and a physical solvent, especially cyclotetramethylene-sulfone. Such systems show good absorption capacity and good selectivity against moderate investment costs and operational costs. They perform very well at high pressures, especially between 20 and 90 bara.

The physical absorption process is preferred for the present application, and is in itself well known to the man skilled in the art. Reference is be made to e.g. Perry, Chemical Engineers' Handbook, Chapter 14, Gas Absorption. The liquid absorbent in the physical absorption process is suitably methanol, ethanol, acetone, dimethyl ether, methyl i-propyl ether, polyethylene glycol or xylene, preferably methanol. This process is based on carbon dioxide and hydrogen sulfide being highly soluble under pressure in the methanol, and then being readily releasable from solution when the pressure is reduced as further discussed below. This high pressure system is preferred due to its efficiency, although other removal systems such as using amines are known. The physical absorption process is suitably carried out at low temperatures, preferably between −60° C. and 0° C., preferably between −30 and −10° C.

The physical absorption process may be carried out by contacting the light products stream in a counter-current upward flow with the liquid absorbent. The absorption process is preferably carried out in a continuous mode, in which the liquid absorbent is regenerated. This regeneration process is well known to the man skilled in the art. The loaded liquid absorbent is suitably regenerated by pressure release (e.g. a flashing operation) and/or temperature increase (e.g. a distillation process). The regeneration is suitably carried out in two or more steps, preferably 3 to 10 steps, especially a combination of one or more flashing steps and a distillation step.

The regeneration of solvent from the process is also known in the art. Preferably, the present invention involves one integrated solvent regeneration tower. Further process conditions are for example described in U.S. Pat. No. 4,142,875 and EP-B-651042, both incorporated herein by reference.

Preferably the gaseous stream or the CO-depleted stream is subjected to one or more further removal systems prior to using said stream in optional step (e). These removal systems may be guard or scrubbing units, either as back-up or support to the CO₂/H₂S removal system, or to assist in the reduction and/or removal of other contaminants such as HCN, NH₃, COS and H₂S, metals, carbonyls, hydrides or other trace contaminants.

Part of the CO-depleted stream may also be used for manufacture of or preparation of hydrogen. This may typically be done in hydrogen separation unit such as, for example, a Pressure Swing Adsorption (PSA) unit, a membrane separation unit or combinations of these. Hydrogen separated this way can then be used as the hydrogen source in the hydrocracking of the hydrocarbon products as made in step (e), in particular when step (e) involves a Fischer-Tropsch synthesis step. This arrangement reduces or even eliminates the need for a separate source of hydrogen, e.g. from an external supply, which is otherwise commonly used where available.

Optional step (e) may involve a single stage or a multi-stage process for the production of the hydrocarbon product(s). Each stage would then have one or more reactors.

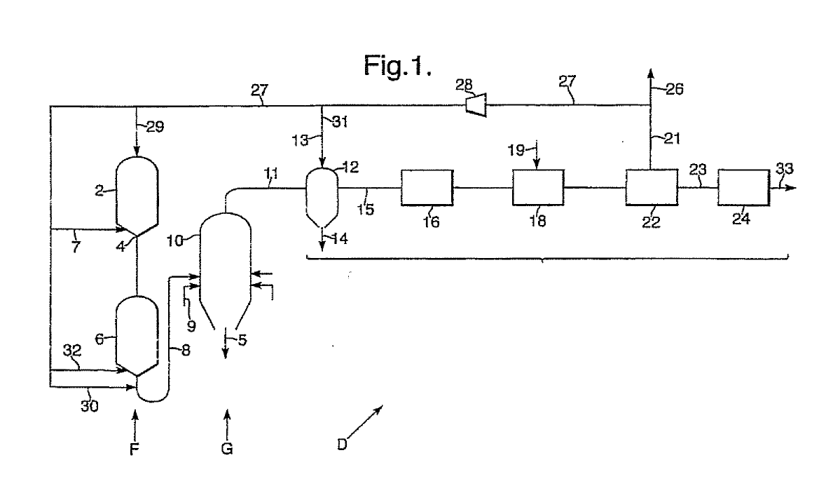

FIG. 1 schematically shows a process block scheme of a carbonaceous fuel to hydrocarbon product synthesis system to carry out process of producing a hydrocarbon product from a carbonaceous fuel such as coal. For simplicity, valves and other auxiliary features are not shown. The system comprises: a carbonaceous fuel supply system (F); a gasification system (G) wherein a gasification process takes place to produce a gaseous stream of an intermediate product containing synthesis gas; and a downstream system (D) for further processing of the intermediate product into the final organic substance that forms the hydrocarbon product. A process path extends through the fuel supply system F and the downstream system D via the gasification system G.

In the described embodiment the fuel supply system F comprises a sluicing hopper 2 and a feed hopper 6. The gasification system G comprises a gasification reactor 10. The fuel supply system is arranged to pass the carbonaceous fuel along the process path into the gasification reactor 10. The downstream system D comprises an optional dry-solids removal unit 12, an optional wet scrubber 16, an optional shift conversion reactor 18, a CO₂ recovery system 22, and a hydrocarbon product synthesis reactor 24 wherein a suitable organic-substance forming reaction can be driven.

Preferred details of these features will be provided hereinafter.

The sluicing hopper 2 is provided for sluicing the dry, solid carbonaceous fuel, preferably in the form of fine particulates of coal, from a first pressure under which the fuel is stored, to a second pressure where the pressure is higher than the first pressure. Usually the first pressure is the natural pressure of about 1 atmosphere, while the second pressure will exceed the pressure under which the gasification process takes place.

In a gasification process, the pressure may be higher than 10 atmospheres. In a gasification process in the form of a partial combustion process, the pressure may be between 10 and 90 atmospheres, preferably between 10 and higher than 70 atmospheres, more preferably 30 and 60 atmospheres.

The term fine particulates is intended to include at least pulverized particulates having a particle size distribution so that at least about 90% by weight of the material is less than 90 μm and moisture content is typically between 2 and 12% by weight, and preferably less than about 5% by weight.

The sluicing hopper discharges into the feed hopper 6 via a discharge opening 4, to ensure a continuous feed rate of the fuel to the gasification reactor 10. The discharge opening 4 is preferably provided in a discharge cone, which in the present case is provided with an aeration system 7 for aerating the dry solid content of the sluicing hopper 2.

The feed hopper 6 is arranged to discharge the fuel via conveyor line 8 to one or more burners provided in the gasification reactor 10. Typically, the gasification reactor 10 will have burners in diametrically opposing positions, but this is not a requirement of the present invention. Line 9 connects the one or more burners to a supply of an oxygen containing stream (e.g. substantially pure O₂ or air). The burner is preferably a co-annular burner with a passage for an oxygen containing gas and a passage for the fuel and the transport gas. The oxygen containing gas preferably comprises at least 90% by volume oxygen. Nitrogen, carbon dioxide and argon being permissible as impurities. Substantially pure oxygen is preferred, such as prepared by an air separation unit (ASU). Steam may be present in the oxygen containing gas as it passes the passage of the burner. The ratio between oxygen and steam is preferably from 0 to 0.3 parts by volume of steam per part of oxygen. A mixture of the fuel and oxygen from the oxygen-containing stream reacts in a reaction zone in the gasification reactor 10.

A reaction between the carbonaceous fuel and the oxygen-containing fluid takes place in the gasification reactor 10, producing a gaseous stream of synthesis gas containing at least CO, CO₂ and H₂. Generation of synthesis gas occurs by partially combusting the carbonaceous fuel at a relatively high temperature somewhere in the range of 1000° C. to 3000° C. and at an elevated pressure. Slag and other solids can be discharged from the gasification reactor via line 5, after which they can be further processed for disposal. As the person skilled in the art is assumed to be familiar with suitable conditions for partially oxidising a carbonaceous fuel thereby obtaining synthesis gas, these conditions are not discussed here in further detail.

The feed hopper 6 preferably has multiple feed hopper discharge outlets, each outlet being in communication with at least one burner associated with the reactor. Typically, the pressure inside the feed hopper 6 exceeds the pressure inside the reactor 10, in order to facilitate injection of the powdered coal into the reactor.

The gaseous stream of synthesis gas leaves the gasification reactor 10 through line 11 at the top, where it is cooled. To this end a syngas cooler (not shown) may be provided downstream of the gasification reactor 10 to have some or most of the heat recovered for the generation of, for instance, high-pressure steam. Eventually, the synthesis gas enters the downstream system D in a downstream path section of the process path, wherein the dry-solids removal unit 12 is optionally arranged.

The dry-solids removal unit 12 may be of any type, including the cyclone type. In the embodiment of FIG. 1, it is provided in the form of a preferred ceramic candle filter unit as for example described in EP-A-551951, incorporated herein by reference. Line 13 is in fluid communication with the ceramic candle filter unit to provide a blow back gas pressure pulse at timed intervals in order to blow dry solid material that has accumulated on the ceramic candles away from the ceramic candles. The dry solid material is discharged from the dry-solids removal unit via line 14 from where it is further processed prior to disposal.

Suitably, the blow back gas for the blow back gas pressure pulse is preheated to a temperature of between 200° C. and 260° C., preferably around 225° C., or any temperature close to the prevailing temperature inside the dry-solid removal unit 12. The blow back gas is preferably buffered to dampen supply pressure effects when the blow back system is activated.

The filtered gaseous stream 15, now substantially free from dry solids, progresses along the downstream path section of the process path through the downstream system, and is fed, optionally via wet scrubber 16 and optional shift conversion reactor 18, to the CO₂-recovery system 22. The CO₂-recovery system 22 functions by dividing the gaseous stream into a CO₂-rich stream and a CO₂ poor (but CO- and H₂-rich) stream and. The CO₂-recovery system 22 has an outlet 21 for discharging the CO₂-rich stream and an outlet 23 for discharging the CO₂-poor stream in the process path. Outlet 23 is in communication with the hydrocarbon product synthesis reactor 24, where the discharged CO₂-poor, H₂-rich stream can be subjected to the desired organic-substance forming reaction.

Depending on the final hydrocarbon product and on the type of hydrocarbon synthesis reactor 24 employed, there is an optimal synthesis gas 10 composition. As stated before, synthesis gas 10 discharged from the gasification reactor comprises at least H₂, CO, and CO₂, in relative abundances dependent on inter alia the type of carbonaceous fuel feedstock employed. The H₂/CO ratio in synthesis gas formed by gasification of most types of carbonaceous fuels defined in the present disclosure is generally about 1 or less than about 1. For coal-derived synthesis gas, the H₂/CO ratio is commonly between 0.3 and 0.6, and for heavy-residue derived synthesis gas it is commonly between 0.5 and 0.9. Throughout the specification the H₂/CO ratio is given as mole ratio.

Depending on the requirements of a subsequent hydrocarbon synthesis process, the composition may be optimised in various ways. The process as exemplified in FIG. 1 comprises optional shift conversion reactor 18 which increases the H₂/CO ratio. Subsequent CO₂-recovery system 22 increases the H₂/CO₂ ratio and produces a CO₂ stream.

Any type of CO₂-recovery may be employed, but absorption based CO₂-recovery is preferred, such as physical or chemical washes, because such recovery also removes sulphur-containing components such as H₂S from the process path.

The CO₂-recovery system 22 can alternatively be located downstream of the hydrocarbon synthesis reactor 24, since a significant fraction of the CO₂ will generally not be converted into the organic substance to be synthesised. Or an additional CO₂-recovery system can be located downstream of the hydrocarbon synthesis reactor 24 in addition to the CO₂-recovery system 22 upstream of reactor 24.

The CO₂-rich stream becomes available for a variety of uses to assist the process, of which examples will now be discussed.

A feedback line 27 is provided to bring a feedback gas from the downstream system D to feedback inlets providing access to one or more other points in the process path that lie upstream of the outlet 21, suitably via one or more of branch lines 7, 29, 30, 31, 32 each being in communication with line 27.

Blowback lines may be provided at the outlet of the gasifier and the inlet of the optional syngas cooler. Such blowback lines, although presently not shown in FIG. 1, would serve to supply blow back gas for clearing local deposits. Line 27 is in communication with outlet 21. Excess CO₂-rich gas may be removed from the cycle via line 26.

A compressor 28 may optionally be provided in line 27 to generally adjust the pressure of the feedback gas. It is also possible to locally adjust the pressure in one or more of the branch lines, as needed, either by pressure reduction or by (further) compression. Another option is to provide two or more parallel feedback lines to be held at mutually different pressures using compression in each of the parallel feedback lines. The most attractive option will depend on the relative consumptions.

Herewith a separate source of compressed gas for bringing additional gas into the process path is avoided. Typically in the prior art, nitrogen is used for instance as the carrier gas for bringing the fuel to and into the gasification reactor 10, or as the blow-back gas in the dry solids removal unit 12 or as purge gas or aeration gas in other places. This unnecessarily brings inert components into the process path, which adversely affects the methanol synthesis efficiency. CO₂ is available from the gaseous stream anyway, and thus one may take advantage of that.

One or more feedback gas inlets are preferably provided in the fuel supply system such that in operation a mixture comprising the carbonaceous fuel and the feedback gas is formed. Herewith an entrained flow of the carbonaceous fuel with a carrier gas containing the feedback gas can be formed in conveyor line 8 to feed the gasification reactor 10. Examples can be found in the embodiment of FIG. 1, where branch lines 7 and 29 discharge into the sluicing hopper 2 for pressurising the sluicing hopper 2 and/or aerating its content, branch line 32 discharges into the feed hopper 6 to optionally aerate its content, and branch line 30 feeds the feedback gas into the conveyor line 8.

The feedback gas is preferably brought into the process path through one or more sintered metal pads, which can for instance be mounted in the conical section of sluicing hopper 2. In the case of conveyor line 8, the feedback gas may be directly injected.

In addition or instead, one or more feedback gas inlets can be provided in the dry-solids removal unit 12 where it can be utilized as blow-back gas.

Again in addition or instead, one or more feedback gas inlets can be provided in the form of purge stream inlets for injecting a purging portion of the feedback gas into the process path to blow dry solid accumulates such as fly ash back into the gaseous steam.

The use of CO₂ as the transport gas will now be further exemplified in the following two examples.

### EXAMPLE 1

The following Table I illustrates, in a line up as shown and described with reference to FIG. 1, the effect of using CO₂ from the CO₂-recovery system 22 for coal feeding and blowback purposes, instead of nitrogen, on the synthesis gas composition. The synthesis gas capacity (CO and H₂) was 72600 NM³/hr, but any other capacity will do as well. The middle column gives the composition of the synthesis gas exiting from wet scrubber 16 when CO₂-rich feedback gas from the CO₂-recovery system 22 was utilized for coal feeding into the gasification reactor 10, and blow back of the dry solids removal unit 12. The right hand column gives a reference where N₂ was used instead of the feedback gas.

TABLE I
composition (in mol. %)

| | CO₂ Feedback gas | N₂ based |
|---|---|---|
| | (embodiment of inv.) | (reference) |
| CO + H₂ | 89.3 | 87.8 |
| CO | 69.6 | 64.1 |
| H₂ | 19.7 | 23.7 |
| N₂ | 0.44 | 4.84 |
| CO₂ | 9.29 | 6.42 |
| H₂S | 0.44 | 0.67 |
| H₂0 | 18.8 | 18.8 |

As can be seen, the nitrogen content in the synthesis gas is decreased by more than a factor of ten utilizing the invention relative to the reference. The CO₂ content has increased a little relative to the reference, but this is considered to be of minor importance relative to the advantage of lowering the nitrogen content because CO₂ does not burden the methanol synthesis reaction or Fischer-Tropsch synthesis as much as nitrogen does. Moreover CO₂ will always be part of the synthesis gas composition, especially after performing a water shift reaction.

### EXAMPLE 2

The following Table II illustrates, in a line up as shown and described with reference to FIG. 1, the effect of using a weight ratio of CO₂ to the solid coal fuel of less than 0.5 (dense phase) according to the invention (T1-T3), as compared with the weight ratio of about 1.0 (dilute phase) as used in the Example I of U.S. Pat. No. 3,976,442. As can be seen from Table II, the oxygen consumption per kg oxygen according to the present invention is significantly lower than the oxygen consumption in case of Example I of U.S. Pat. No. 3,976,442. Preferably the weight ratio of CO₂ to coal is between 0.12 and 0.20.

TABLE II
influence of weight ratio of CO₂ to the
carbonaceous fuel

| | | | | Example I of |
|---|---|---|---|---|
| | | | | U.S. Pat. No. |
| | T1 | T2 | T3 | 3 976 442 |
| Weight | 0.14 | 0.19 | 0.29 | 1.0 |
| ratio of | | | | |
| CO₂ to coal | | | | |
| CO + H₂ | 95.8 | 89.9 | 87.6 | 83.76 |
| [mol %] | | | | |
| CO [mol %] | 77.3 | 72.0 | 72.2 | 67.46 |
| H₂ [mol %] | 18.5 | 17.9 | 15.4 | 16.30 |
| N₂ [mol %] | 0.5 | 0.4 | 0.4 | 0.58 |
| CO₂ [mol %] | 1.8 | 4.8 | 6.4 | 13.03 |
| H₂S [mol %] | 0.1 | 0.1 | 0.1 | 1.65 |
| H₂O [mol %] | 1.7 | 4.6 | 5.3 | Not |
| | | | | indicated |
| O₂/Coal | 0.734 | 0.748 | 0.758 | 0.901 |
| [kg/kg] | | | | |

Having concluded these examples, it is remarked that the feedback inlets can be connected to an external gas supply, for instance for feeding in CO₂ or N₂ or another suitable gas during a start-up phase of the process. When a sufficient amount of syngas—and accordingly a sufficient amount of CO₂— is being produced, the feedback inlet may then be connected to the outlet arranged to discharge the feedback gas containing CO₂ from the internally produced CO₂-rich stream. Preferably nitrogen is used as external gas for start-up of the process. Normally in start-up situations, no carbon dioxide will be readily available. When the amount of carbon dioxide as recovered from the gaseous stream prepared in step (b) is sufficient, the amount of nitrogen can be reduced to zero. Nitrogen is suitably prepared in a so-called air separation unit which unit also prepares the oxygen-containing stream of step (a). The invention is thus also related to a method to start the process according to a specific embodiment of the invention wherein the carbon dioxide as obtained in step (d) is used in step (a). In this method nitrogen is used as transport gas in step (a) until the amount of carbon dioxide as obtained in step (d) is sufficient to replace the nitrogen.

It has been stated above that, depending on the final hydrocarbon product and on the type of hydrocarbon synthesis reactor 24 employed, there is an optimal synthesis gas 10 composition.

In the case that the desired hydrocarbon product is methanol, the organic-substance forming reaction would be a methanol-forming reaction whereby reactor 24 would be a methanol synthesis reactor.

However, reactor 24 may also comprise a Fischer-Tropsch reactor which is capable of forming other hydrocarbon products as will be discussed in more detail later in this specification.

The suitability of the synthesis gas composition for the methanol forming reaction is expressed as the stoichiometric number SN of the synthesis gas, whereby expressed in the molar contents [H₂], [CO], and [CO₂], SN=([H₂]−[CO₂])/([CO]+[CO₂]). It has been found that the stoichiometric number of the synthesis gas produced by gasification of the carbonaceous feed is lower than the desired ratio of about 2.05 for forming methanol in the methanol synthesis reactor 24. By performing a water shift reaction in shift conversion reactor 18 and separating part of the carbon dioxide in CO₂-recovery system 22 the SN number can be improved. Preferably hydrogen separated from the methanol synthesis off gas can be added to the synthesis gas to further increase the SN (not shown in Figure).

Therefore, an advantage of an upstream location relative to the methanol synthesis reactor 24 is that the CO- and H₂-rich stream forms an improved starting mixture for a subsequent methanol synthesis reaction, because it has an increased stoichiometric ratio—defined as ([H₂]−[CO₂])/([CO]+[CO₂]) wherein [X] signifies the molar content of molecule X whereby X is H₂, CO, or CO₂-closer to the optimal stoichiometric number of about 2.05 for the synthesis of methanol.

In the embodiment of FIG. 1, an optional shift conversion reactor 18 is disposed in the process path upstream of the CO₂-recovery system 22. The shift conversion reactor is arranged to convert CO and Steam into H₂ and CO₂. Steam can be fed into the shift conversion reactor via line 19. An advantage hereof is that the amount of H₂ in the gaseous mixture is increased so that the stoichiometric ratio is further increased. The CO₂ as formed in this reaction may be advantageously used as transport gas in step (a).

The methanol that is discharged from the methanol synthesis reactor 24 along line 33 may be further processed to meet desired requirements, for instance including purification steps that may include for instance distillation, or even including conversion steps to produce other liquids such as for instance one or more of the group including gasoline, dimethyl ether (DME), ethylene, propylene, butylenes, isobutene and liquefied petroleum gas (LPG).

Hydrocarbon synthesis reactor 24 may also comprise a Fischer-Tropsch reactor for carrying out Fischer-Tropsch synthesis. The Fischer-Tropsch synthesis is well known to those skilled in the art and involves synthesis of hydrocarbons from a gaseous mixture of hydrogen and carbon monoxide, by contacting that mixture at reaction conditions with a Fischer-Tropsch catalyst.

The Fischer-Tropsch synthesis reaction may comprise a single-stage or a multi-stage Fischer-Tropsch process, in particular a two-stage Fischer-Tropsch process.

Products of the Fischer-Tropsch synthesis may range from methane to heavy paraffinic waxes. Preferably, the production of methane is minimised and a substantial portion of the hydrocarbons produced have a carbon chain length of a least 5 carbon atoms. Preferably, the amount of C₅₊ hydrocarbons is at least 60% by weight of the total product, more preferably, at least 70% by weight, even more preferably, at least 80% by weight, most preferably at least 85% by weight. Reaction products which are liquid phase under reaction conditions may be physically separated Gas phase products such as light hydrocarbons and water may be removed using suitable means known to the person skilled in the art.

Fischer-Tropsch catalysts are known in the art, and typically include a Group VIII metal component, preferably cobalt, iron and/or ruthenium, more preferably iron and cobalt. The Fischer-Tropsch synthesis may be carried out in a multi-tubular reactor, a slurry phase regime or an ebullating bed regime, wherein the catalyst particles are kept in suspension by an upward superficial gas and/or liquid velocity.

The reaction in hydrocarbon-synthesis reactor 24 may be formed by an iron catalyzed Fischer-Tropsch synthesis reaction. Such reaction may be performed in a slurry phase reactor or in an ebullating bed regime. Examples of iron based catalysts and processes are the commercial Sasol process as operated in South Africa and those described in for example US-A-20050203194, US-A-20050196332, U.S. Pat. No. 6,976,362, U.S. Pat. No. 6,933,324 and EP-A-1509323, all of which are incorporated herein by reference. In case a cobalt based catalyst is used to make a very heavy Fischer-Tropsch wax product it is found desirable to use a multi-tubular reactor.

Typically, the catalysts comprise a catalyst carrier. The catalyst carrier is preferably porous, such as a porous inorganic refractory oxide, more preferably alumina, silica, titania, zirconia or mixtures thereof.

The optimum amount of catalytically active metal present on the carrier depends inter alia on the specific catalytically active metal. Typically, the amount of cobalt present in the catalyst may range from 1 to 100 parts by weight per 100 parts by weight of carrier material, preferably from 10 to 50 parts by weight per 100 parts by weight of carrier material.

The catalytically active metal may be present in the catalyst together with one or more metal promoters or co-catalysts. The promoters may be present as metals or as the metal oxide, depending upon the particular promoter concerned. Suitable promoters include oxides of metals from Groups IIA, IIIB, IVB, VB, VIIB and/or VIIB of the Periodic Table, oxides of the lanthamides and/or the actinides. Preferably, the catalyst comprises at least one of an element in Group IVB, VB and/or VIIB of the Periodic Table, in particular titanium, zirconium, manganese and/or vanadium. As an alternative or in addition to the metal oxide promoter, the catalyst may comprise a metal promoter selected from Groups VIIB and/or VIII of the Periodic Table. Preferred metal promoters include rhenium, platinum and palladium.

Reference to “Groups” and the “Periodic Table” as used herein relate to the “previous IUPAC form” of the Periodic Table such as that described in the 68^{th} edition of the Handbook of Chemistry and Physics (CPC Press).

A most suitable catalyst comprises cobalt as the catalytically active metal and zirconium as a promoter. Another most suitable catalyst comprises cobalt as the catalytically active metal and manganese and/or vanadium as a promoter.

The promoter, if present in the catalyst, is typically present in an amount of from 0.1 to 60 parts by weight per 100 parts by weight of carrier material. It will however be appreciated that the optimum amount of promoter may vary for the respective elements which act as promoter. If the catalyst comprises cobalt as the catalytically active metal and manganese and/or vanadium as promoter, the cobalt:(manganese+vanadium) atomic ratio is advantageously at least 12:1.

The Fischer-Tropsch synthesis is preferably carried out at a temperature in the range from 125 to 350° C., more preferably 175 to 275° C., most preferably 200 to 260° C. The pressure preferably ranges from 5 to 150 bar abs., more preferably from 5 to 80 bar abs.

A shift reaction 18 such as exemplified in FIG. 1 may be advantageous as well in order to increase the H₂/CO ratio. This may be especially true when the Fischer-Tropsch reaction is aided by a cobalt-based catalyst, but also iron-based catalysed Fischer-Tropsch processes are known which operate at higher H₂/CO ratios.

A suitable source for the water required in the shift reaction is the product water produced in the Fischer-Tropsch reaction. Preferably this is the main source, e.g. at least 80% is derived from the Fischer-Tropsch process, preferably at least 90%, more preferably 100%. Thus the need of an external water source is minimised. Some Fischer-Tropsch processes, including many iron-based Fischer-Tropsch processes, allow for a lower H₂/CO ratio, in which case the optional shift conversion step may be omitted.

But when the optional shift conversion step 18 is used in conjunction with a Fischer-Tropsch process, it is generally preferred to arrive at a H₂/CO ratio of the CO-depleted stream of greater than 1.4, and preferably greater than 1.5. A suitable target H₂/CO ratio lies between 1.4 and 1.95, more preferably in the range 1.6 to 1.9, and even more preferably in the range 1.6 to 1.8.

The water shift conversion reaction may produce a highly enriched synthesis gas, possibly having a H₂/CO ratio above 3, above 5, above 7, above 15, or even above 20 or more. Such high ratios typically can be obtained in catalytic water shift reaction processes.

In order to arrive at the desired H₂/CO ratio for the subsequent desired hydrocarbon synthesis step, it is possible to perform the shift conversion of step (d) only on part of the gaseous stream obtained in step (b) or (c). In such a case, the gaseous stream of step (b) or (c) is divided into at least two sub-streams, one of which undergoes step (d) conversion to obtain a first CO-depleted stream. This first CO-depleted stream, or at least part thereof, is then combined with one or more of the other sub-streams to form a second CO-depleted sub-stream. If desired or necessary, one or more of the sub-streams that are not subjected to step (d) could be used for other parts of the process, rather than being combined with the shift-converted CO-depleted sub-streams. Examples of other uses include generation of steam or power.

When a multi-stage hydrocarbon synthesis step is used, e.g. a multi-stage or two stage Fischer-Tropsch process, part of the CO-depleted stream may be employed as additional feed for one or more of the further stages.

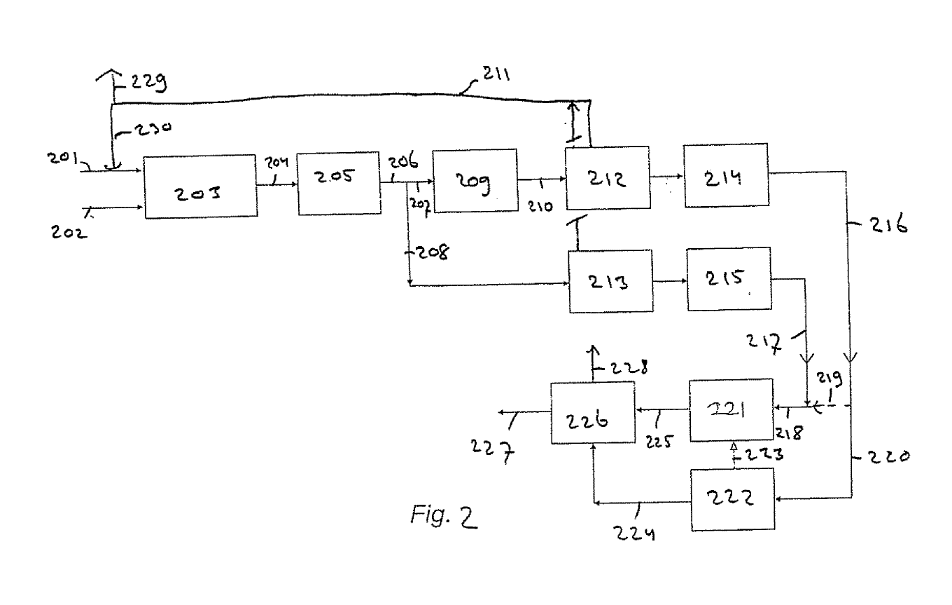

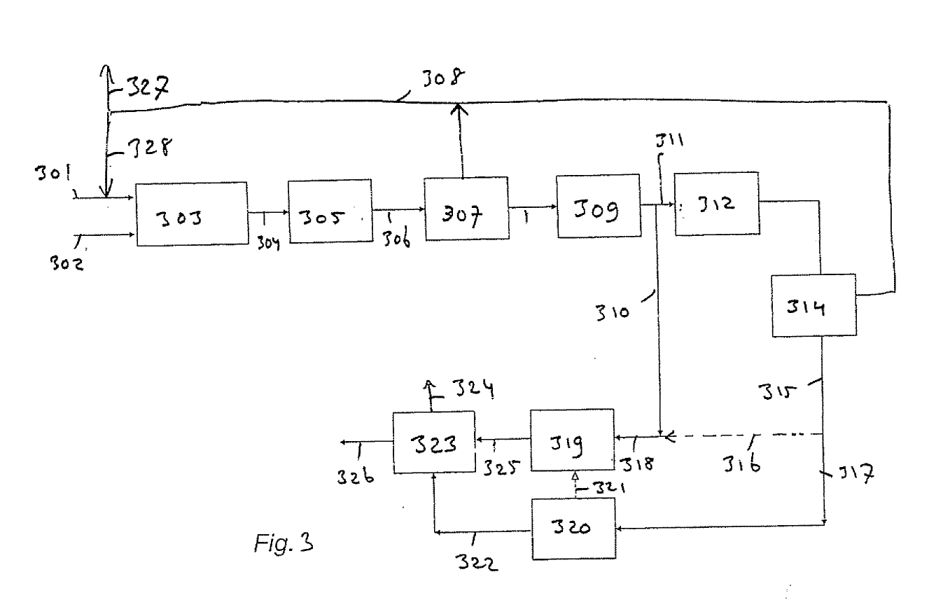

FIGS. 2 and 3 illustrate such embodiments, wherein only part of the gaseous stream obtained from step (d) is shift converted.

Turning to FIG. 2, there is shown a process for the synthesis of hydrocarbons from coal. This starts with gasification in a gasification reactor 203 of coal 201 with oxygen 202 to form a synthesis gas stream 204, followed by removal of solids such as slag and soot and the like in a step 205. Step 205 is a schematic representation of the dry-solids removal unit 12 and scrubber 16 of FIG. 1, while line 204 in FIG. 2 corresponds to line 11 in FIG. 1. The synthesis gas stream 206 is then divided into two sub-streams 207 and 208. Sub-stream 208 is a ‘by-pass’ stream, which passes through a CO₂/H₂S removal system 213 followed by one or more guard beds and/or scrubbing units 215 to provide a cleaned sub-stream 217. The units 215 serve as backup or support to the CO₂/H₂S removal system 213, or to assist in the reduction and/or removal of other contaminants such as HCN, NH₃, COS and H₂S.

The other sub-stream 207 of synthesis gas passes into a sour shift unit 209 to undergo a catalytic water shift conversion reaction wherein the H₂/CO ratio is significantly increased, optionally in a manner known in the art. The gas stream from the sour shift unit then undergoes the same or similar CO₂/H₂S removal in unit 212, followed by the same or similar guard beds 214 as the synthesis gas stream 208. A first CO depleted stream 216 is obtained. Carbon dioxide as separated may be fed to carbon dioxide discharge line 211. At least part 230 of the CO₂ is fed into line 201 to be used as transport gas and any excess CO₂ 229 may be used otherwise, e.g. such as shown above with reference FIG. 1.

The first CO-depleted synthesis gas stream 216 may be re-combined via stream 219 with the non-converted, cleaned synthesis gas sub-stream 217 in case the Fischer-Tropsch process is a cobalt catalyzed based process. In case of an iron based Fischer-Tropsch process the first CO-depleted stream 216 does not necessarily need to be combined. Instead, stream 216 may be used as feed 220 to a hydrogen purification unit 222 from which purified hydrogen streams 223 and 224 are discharged. A second CO-depleted stream 218 is used as feed to a hydrocarbon synthesis reactor system 221, which may involve one or more reactors or units in one or more stages.

The proportion of the first CO-depleted stream that may advantageously be used for hydrogen manufacture is less than 10% by volume, in particular approximately 1 to 7% by volume as calculated on the first CO-depleted stream 216. Such hydrogen may be used in an upgrading unit 226 as will now be illustrated.

In system 221 a hydrocarbon product 225 is obtained which may be further processed in upgrading unit 226. When the hydrocarbon synthesis system 221 includes a Fischer-Tropsch reactor, upgrading unit 226 may be employed to obtain among other products a middle distillate 227, like kerosene and gas oil. Unit 226 may then involve flashing, distillation, hydrogenation and hydroconversion, like hydrocracking, hydroisomerisation and catalytic dewaxing. A Fischer-Tropsch off-gas 228 will be obtained from which carbon dioxide can be isolated.

Hydrogen 223 and 224 as prepared in unit 222 may be used in the Fischer-Tropsch or methanol synthesis, and preferably in the various hydroprocessing steps of unit 226.

FIG. 3 shows a similar process as FIG. 2. The following reference numbers of FIG. 3 have the meaning of the respective reference numbers of FIG. 2: 301 is as 201; 302 is as 202; 303 is as 203; 304 is as 204; 305 is as 205; line 306 is as line 206; 325 is as 225; 323 is as 226; 326 is as 227; 324 is as 228; 308 is as 211; 327 is as 229; and 328 is as 230.

However, in the process shown in FIG. 3 as compared to that of FIG. 2, an additional CO₂/H₂S removal unit 307 provides the CO₂/H₂S cleaning of the synthesis gas stream 306 prior to division into sub-streams 311 and 310.

After the CO₂/H₂S removal unit 307 and guard beds 309, the synthesis gas stream is then divided into 311 and 310, such that sub-stream 310 passes directly towards the hydrocarbon synthesis system 319. Meanwhile, the other divided synthesis gas sub-stream 311 undergoes a sweet shift conversion 312, followed by subsequent CO₂/H₂S cleaning 314, which should not need to treat for H₂S. The converted sweet shift stream 315 (first CO-depleted stream) may then be wholly or substantially combined with the—non-converted—by-pass sub-stream 310 to provide a synthesis gas stream 318 entering the hydrocarbon product synthesis reactor 319 with an enhanced the H₂/C0 ratio as desired for the type of hydrocarbon product synthesis reactor employed. This may be a cobalt based Fischer-Tropsch reactor or a methanol synthesis reactor.

In case of an iron-based Fischer-Tropsch process 319, line 316 may sometimes be omitted as explained above.

Like FIG. 2, a part or all of the first CO-depleted stream 317 could be supplied to a hydrogen purification unit 320 to make hydrogen streams 321 and 322.

The division of the gaseous stream into sub-streams, before subjecting it to a shift conversion, facilitates creation of any desired H₂/CO ratio following their recombination. Any degree or amount of division is possible. Where the gaseous stream is divided into two sub-streams, the division into the sub-streams could be in the range 80:20 to 20:80 by volume, preferably 70:30 to 30:70 by volume, depending upon the desired final H₂/CO ratio. Simple analysis of the H₂/CO ratios in the second CO-depleted stream and knowledge of the desired ratio allows easy calculation of the division. In the case that one stream is to be used as feed for e.g. a second stage of a Fischer-Tropsch process in step (e), this stream will usually be between 10 and 50%, preferably between 20 and 35% of the first CO depleted stream.

The ability to change the degree of division into the sub-streams also provides a simple but effective means of accommodating variations in the H₂/CO ratio in the gaseous stream as obtained in step (b) which variations are primarily due to variations in feedstock quality. With feedstock quality is here meant especially the hydrogen and carbon content of the original fuel, for example, the ‘grade’ of coal. Certain grades of coal generally having a higher carbon content, but a high carbon content, will, after gasification of the coal, provide a greater production of carbon monoxide, and thus a lower H₂/CO ratio. However, using other grades of coal means removing more contaminants or unwanted parts of the coal, such as ash and sulfur and sulfur-based compounds. The ability to change the degree of division of the fuel-derived syngas stream into the sub-streams allows the process to use a variety of fuel feedstocks, generally ‘raw’ coal, without any significant re-engineering of the process or equipment to accommodate expected or unexpected variations in such coals.

Clearly, the CO₂ recovery may be performed on the gaseous stream obtained in step (b), on the sub-streams as obtained from the gaseous stream of step (b) or on the combined second CO-depleted stream. Preferably the CO₂ recovery is as part of step (d) subsequent to a shift conversion. More preferably the CO₂ recovery from the sub-stream, which stream is not being subjected to shift conversion, is performed separately from the CO₂ recovery from the first CO-depleted stream before said streams are combined.

The invention has here been illustrated in accordance with a coal-to-methanol process and system and a Fischer-Tropsch process driven by a hydro-carbonaceous or carbonaceous fuel including coal. The invention is applicable to synthesis of hydroxygenated alkanes in general, including alcohols, methanol, dimethyl ether (DME), or synthesis of alkanes and oxygenated alkanes, which may be formed by subjecting the gaseous stream of synthesis gas to for instance a Fischer-Tropsch reaction.

In particular, the invention also provides one or more process advantages in the manufacturing of H₂. The person skilled in the art will understand that for H₂ manufacturing the hydrocarbon-product forming reactor 24, such as the methanol-forming reactor or the Fischer-Tropsch reactor system is not necessary, but instead there may be a H₂ separator for separating an H₂-rich gas from the synthesis gas stream. Examples of an H₂ separator are a pressure swing adsorber (PSA), a membrane-separator or a cold box separator or combinations of said processes. An advantage of a PSA is that the separated H₂ is readily available at elevated pressure.

We claim:

1. A process for producing synthesis gas from a carbonaceous fuel, the process comprising the steps of:
(a) supplying a carbonaceous fuel and an oxygen-containing stream to a burner of a gasification reactor, wherein a CO₂-containing transport gas is used to transport the solid carbonaceous fuel to the burner whereby the weight ratio of CO₂ to the carbonaceous fuel is less than 0.5 on a dry basis;
(b) partially oxidising the carbonaceous fuel in the gasification reactor, thereby obtaining a gaseous stream comprising CO, CO₂, and H₂; and
(c) removing the gaseous stream obtained in step (b) from the gasification reactor.

2. The process according to claim 1, wherein the CO₂-containing stream used in step (a) is supplied at a velocity of less than 20 m/s.

3. The process according to claim 1, wherein the weight ratio in step (a) is in the range from 0.12-0.49 on a dry basis.

4. The process according to claim 1, wherein the CO₂-containing transport gas used in step (a) comprises at least 80% CO₂.

5. The process according to claim 1, wherein the solid carbonaceous fuel comprises coal.

6. The process according to claim 1, wherein the gaseous stream removed from the gasification reactor in step (c) comprises from 1 to 10 mol % CO₂ on a dry basis.

7. The process according to claim 1, wherein the gaseous stream removed from the gasification reactor in step (c) is further processed.

8. The process according to claim 7, wherein the further processing comprises the step of:
(d) shift converting the gaseous stream removed from the gasification reactor in step (c) by at least partially converting CO into CO₂, thereby obtaining a CO-depleted stream.

9. The process according to claim 8, wherein step (d) further comprises subjecting the CO-depleted stream to a CO₂-recovery system thereby obtaining a CO₂-rich stream and a CO₂-poor, CO-depleted stream.

10. The process according to claim 9, wherein the CO₂-recovery system is a combined carbon dioxide and hydrogen sulphide removal system.

11. The process according to claim 9, wherein the CO₂-recovery system is based on a physical solvent process.

12. The process according to claim 11, wherein the physical solvent is methanol.

13. The process according to claim 9, wherein at least part of the CO₂-rich stream is used in the CO₂-containing transport gas as used in step (a).

14. The process according to claim 13, wherein nitrogen is used as transport gas in step (a) until the amount of carbon dioxide as obtained in step (d) is sufficient to replace the nitrogen.

15. The process according to claim 1, wherein the mole ratio of hydrogen to CO in the gaseous stream of step (c) is less than about 1.

16. A process for producing a hydrocarbon product from a carbonaceous fuel, the process comprising the steps of:
(a) supplying a carbonaceous fuel and an oxygen-containing stream to a burner of a gasification reactor, wherein a CO₂-containing transport gas is used to transport the solid carbonaceous fuel to the burner and wherein the weight ratio of CO₂ to the carbonaceous fuel is less than 0.5 on a dry basis;
(b) partially oxidising the carbonaceous fuel in the gasification reactor, thereby obtaining a gaseous stream comprising CO, CO₂, and H₂;
(c) removing the gaseous stream obtained in step (b) from the gasification reactor; and
(e) subjecting the gaseous stream removed from the gasification reactor in step (c) to a hydrocarbon forming reaction wherein said gaseous stream is reacted to form a hydrocarbon product.

17. The process according to claim 16, wherein the CO₂-containing stream used in step (a) is supplied at a velocity of less than 20 m/s.

18. The process according to claim 16, wherein the weight ratio in step (a) is in the range from 0.12-0.49 on a dry basis.

19. The process according to claim 16, wherein the CO₂-containing transport gas used in step (a) comprises at least 80% CO₂.

20. The process according to claim 16, wherein the solid carbonaceous fuel comprises coal.

21. The process according to claim 16, wherein the gaseous stream removed from the gasification reactor in step (c) comprises from 1 to 10 mol % CO₂ on a dry basis.

22. The process according to claim 16, further comprising the step of:
(d) shift converting at least part of the gaseous stream removed from the gasification reactor in step (c) by at least partially converting CO into CO₂, thereby obtaining a CO-depleted stream and subjecting at least part of the CO-depleted stream to the hydrocarbon forming reaction.

23. The process according to claim 22, wherein the mole ratio of hydrogen to CO in the CO-depleted stream is greater than 1.4.

24. The process according to claim 22, wherein step (d) further comprises subjecting the CO-depleted stream to a CO₂-recovery system thereby obtaining a CO₂-rich stream and a CO₂-poor, CO-depleted stream of which at least part is subjected to the hydrocarbon forming reaction.

25. The process according to claim 24, wherein the CO₂-recovery system is a combined carbon dioxide and hydrogen sulphide removal system.

26. The process according to claim 24, wherein the CO₂-recovery system is based on a physical solvent process.

27. The process according to claim 26, wherein the physical solvent is methanol.

28. The process according to claim 24, wherein at least part of the CO₂-rich stream is used in the CO₂-containing transport gas as used in step (a).

29. The process according to claim 22, wherein the gaseous stream removed from the gasification reactor in step (c) is divided into at least two sub-streams, wherein at least one of the sub-streams undergoes step (d) to obtain a first CO-depleted stream, and wherein at least part of the first CO-depleted stream is combined with at least one other of the two sub-streams to form a second CO-depleted stream of which at least a part is subjected to the hydrocarbon forming reaction.

30. The process according to claim 29, wherein the ratio of the part of the gaseous stream that undergoes step (d) versus the part that does not undergo step (d) is in the range 70:30 to 30:70 by volume.

31. The process according to claim 29, wherein the ratio of the part of the gaseous stream that undergoes step (d) versus the part that does not undergo step (d) is in the range 80:20 to 20:80 by volume.

32. The process according to claim 29, wherein the hydrocarbon forming reaction is performed in a multi-stage process whereby part of the first CO-depleted stream is used as an additional feed for one or more of the further stages in the multi-stage process.

33. The process according to claim 16, wherein the mole ratio of hydrogen to CO in the gaseous stream of step (c) is less than about 1.

34. The process according to claim 16, wherein the hydrocarbon forming reaction is performed in a multi-stage process.

35. The process according to claim 16, wherein the hydrocarbon forming reaction is performed in a two-stage process.

36. The process according to claim 22, wherein the hydrocarbon forming reaction is a methanol-forming reaction.

37. The process according to claim 36, wherein the methanol formed in the methanol-forming reaction is subsequently converted to form at least one of the group consisting of gasoline, dimethyl ether, ethylene, propylene, butylenes, isobutene and liquefied petroleum gas.

38. The process according to claim 36, wherein the stoichiometric number of the gas subjected to the methanol-forming reaction lies between 1 and 3.

39. The process according to claim 36, wherein the stoichiometric number of the gas subjected to the methanol-forming reaction lies between 1.9 and 3.

40. The process according to claim 16, wherein the hydrocarbon forming reaction is a Fischer-Tropsch reaction.

41. The process according to claim 40, wherein the product obtained from the Fischer-Tropsch reaction is further subjected to a hydroprocessing step to obtain a hydrocarbon product in the form of a middle distillate fuel.

42. The process according to claim 40, wherein the Fischer-Tropsch reaction comprises an iron-catalysed synthesis step.

43. The process according to claim 42, wherein the iron-catalysed synthesis is conducted in a slurry-phase reactor.

44. The process according to claim 40, wherein the Fischer-Tropsch reaction comprises a cobalt-based catalysis step.

45. The process according to claim 22, wherein the hydrocarbon forming reaction is a Fischer-Tropsch reaction.

46. The process according to claim 45, wherein the mole ratio of hydrogen against CO in the CO-depleted stream is between 1.4 and 1.95.

47. The process according to claim 45, wherein the Fischer-Tropsch reaction produces water in addition to the hydrocarbon product, the process further comprising feeding at least part of the water produced in the Fischer-Tropsch reaction into the shift-converting step.

48. The process according to claim 45, wherein the product obtained from the Fischer-Tropsch reaction is further subjected to a hydroprocessing step to obtain a hydrocarbon product in the form of a middle distillate fuel, and wherein hydrogen is obtained from part of the CO-depleted stream and used in the hydroprocessing step.
